(19) United States
(12) Patent Application Publication
(10) Pub. No.: US 2021/0383928 A1
(43) Pub. Date: Dec. 9, 2021

(54) APPARATUS AND METHOD FOR ESTIMATING BIO-INFORMATION

(71) Applicant: Samsung Electronics Co., Ltd., Suwon-si (KR)

(72) Inventors: Sang Kyu Kim, Yongin-si (KR); Yoon Jae Kim, Seoul (KR); Hyun Seok Moon, Hwaseong-si (KR); Jin Young Park, Hwaseong-si (KR); Sung Mo Ahn, Yongin-si (KR); Kun Sun Eom, Yongin-si (KR); Myoung Hoon Jung, Bucheon-si (KR)

(73) Assignee: Samsung Electronics Co., Ltd., Suwon-si (KR)

(21) Appl. No.: 16/950,258

(22) Filed: Nov. 17, 2020

(30) Foreign Application Priority Data
Jun. 5, 2020 (KR) 10-2020-0068452

(51) Int. Cl. G16H 50/30; A61B 5/00; A61B 5/145

(52) U.S. Cl. CPC G16H 50/30; A61B 5/0075; A61B 5/14546; A61B 5/14532; A61B 5/7475

(57) ABSTRACT

An apparatus for estimating bio-information is provided. The apparatus for estimating bio-information may a spectrometer configured to measure a spectrum from an object of the user; and a processor configured to control an output interface to output guide information to guide the user regarding spectrum measurement based on a hemoglobin index of the user while the spectrum is being measured by the spectrometer; and estimate the bio-information of the user based on a melanin index of the user and the spectrum.

### CROSS-REFERENCE TO RELATED APPLICATION(S)

This application is based on an claims priority under 35 U.S.C. § 119 to Korean Patent Application No. 10-2020-0068452, filed on Jun. 5, 2020, in the Korean Intellectual Property Office, the disclosure of which is incorporated by reference herein in its entirety.

### BACKGROUND

### 1. Field

The following description relates to technology for estimating bio-information from a spectrum of an object.

### 2. Description of Related Art

Recently, methods of non-invasively measuring bio-information, such as a blood glucose and carotenoids, using Raman spectroscopy or near-infrared (NIR) spectroscopy have been studied. Generally, a bio-information measurement instrument using spectroscopic techniques is comprised of a light source for emitting light toward a target object and a detector for detecting an optical signal received from the target object. The bio-information measurement instrument reconstructs a spectrum using the optical signal detected by the detector and measures bio-information through analysis of the reconstructed spectrum.

In general, factors determining skin color may include hemoglobin, carotene, melanin, etc. Particularly, melanin absorbs light very readily and causes degradation of performance, such as a signal-to-noise ratio of a spectrum-based sensor. Accordingly, there have been attempts to analyze data according to skin color, and Fitzpatrick skin type classification including 6 skin types is one of the skin color classification methods. In the case of the Fitzpatrick skin type classification, however, a person subjectively assesses skin color of an object from appearance of the object, and the Fitzpatrick classification is intended to classify skin types according to original skin color and skin sunburn caused by ultraviolet (UV) rays, and hence is not suitable for quantitative analysis of spectrum. In addition, since a person's palm color is bright regardless of race or skin color, the Fitzpatrick skin types are in low correlation with the skin color of the palm.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

According to an aspect of an example embodiment, an apparatus for estimating bio-information of a user may include a spectrometer configured to measure a spectrum from an object of the user; and a processor configured to control an output interface to output guide information to guide the user regarding spectrum measurement based on a hemoglobin index of the user while the spectrum is being measured by the spectrometer; and estimate the bio-information of the user based on a melanin index of the user and the spectrum.

The processor may acquire the hemoglobin index of the user from the spectrum while the spectrum is being measured.

The processor may control the output interface to output the guide information to guide the user regarding a measurement pressure between the object and the spectrometer based on the acquired hemoglobin index.

The processor may control the output interface to output the guide information to guide the user regarding the measurement pressure based on a predefined relationship between the measurement pressure and the hemoglobin index such that the hemoglobin index is maintained to be less than or equal to a predetermined threshold.

The processor may, based on the spectrum being measured, acquire the melanin index from the spectrum.

The processor may, based on the spectrum being measured, acquire a bio-information-related feature from the spectrum.

The processor may estimate the bio-information by applying a predetermined model to the bio-information-related feature based on the melanin index.

The processor may apply a first model to the bio-information-related feature based on the melanin index being greater than or equal to a predetermined threshold, and apply a second model to the bio-information-related feature based on the melanin index being less than the predetermined threshold.

The spectrometer comprises one or more light sources configured to emit light to the object, and one or more detectors configured to detect light scattered or reflected from the object.

The apparatus may include the output interface configured to output a processing result of the processor.

The apparatus may include a communication interface configured to transmit a processing result of the processor to an external device.

The bio-information may include one or more of carotenoids, blood glucose, sugar intake, triglycerides, cholesterol, calories, proteins, body water, extracorporeal water, and uric acid.

A method of estimating bio-information of a user may include measuring a spectrum from an object of the user; guiding a user regarding spectrum measurement based on a hemoglobin index of the user while the spectrum is being measured; and estimating the bio-information of the user based on a melanin index of the user and the spectrum.

The method may include acquiring the hemoglobin index from the spectrum while the spectrum is being measured.

The guiding the user regarding the spectrum measurement comprises guiding the user regarding a measurement pressure between the object and the spectrometer based on the acquired hemoglobin index.

The guiding the user regarding the spectrum measurement may include guiding the user regarding the measurement pressure based on a predefined relationship between the measurement pressure and the hemoglobin index such that the hemoglobin index is maintained to be less than or equal to a predetermined threshold.

The method may include, based on the spectrum being measured, acquiring the melanin index from the spectrum.

The method may include, based on the spectrum being measured, acquiring a bio-information-related feature from the spectrum.

The estimating of the bio-information of the user may include estimating the bio-information of the user by applying a predetermined model to the bio-information-related feature according to the melanin index.

The estimating of the bio-information of the user may include applying a first model to the bio-information-related feature based on the melanin index being greater than or equal to a predetermined threshold, and, applying a second model to the bio-information-related feature based on the melanin index being less than the predetermined threshold.

Other features and aspects will be apparent from the following detailed description, the drawings, and the claims.

### BRIEF DESCRIPTION OF THE DRAWINGS

The above and other aspects, features, and advantages of certain embodiments of the present disclosure will be more apparent from the following description taken in conjunction with the accompanying drawings, in which:

FIG. 1 is a block diagram illustrating an apparatus for estimating bio-information according to an embodiment;

FIG. 2 is a diagram schematically illustrating an embodiment of a structure of a spectrometer;

FIGS. 3A to 3G are diagrams for describing estimation of bio-information;

FIG. 4 is a block diagram illustrating an apparatus for estimating bio-information according to another embodiment;

FIG. 5 is a flowchart illustrating a method of estimating bio-information according to an embodiment; and

FIG. 6 is a diagram illustrating a wearable device according to an embodiment.

Throughout the drawings and the detailed description, unless otherwise described, the same drawing reference numerals will be understood to refer to the same elements, features, and structures. The relative size and depiction of these elements, features, and structures may be exaggerated for clarity, illustration, and convenience.

### DETAILED DESCRIPTION

Details of exemplary embodiments are provided in the following detailed description with reference to the accompanying drawings. The disclosure may be understood more readily by reference to the following detailed description of exemplary embodiments and the accompanying drawings. The disclosure may, however, be embodied in many different forms and should not be construed as being limited to the embodiments set forth herein. Rather, these embodiments are provided so that the disclosure will be thorough and complete and will fully convey the concept of the present disclosure to those skilled in the art, and the disclosure will only be defined by the appended claims. Like reference numerals refer to like elements throughout the specification.

It will be understood that, although the terms “first,” “second,” etc. may be used herein to describe various elements, these elements should not be limited by these terms. These terms are used to distinguish one element from another. Also, the singular forms of terms are intended to include the plural forms of the terms as well, unless the context clearly indicates otherwise. In the specification, unless explicitly described to the contrary, the word “comprise,” and variations such as “comprises” or “comprising,” will be understood to imply the inclusion of stated elements but not the exclusion of any other elements. Terms such as “unit” and “module” denote units that process at least one function or operation, and the units may be implemented by using hardware, software, or a combination of hardware and software.

Hereinafter, embodiments of an apparatus and method for estimating bio-information will be described in detail with reference to the drawings.

Various embodiments of the apparatus for estimating bio-information may be mounted in a variety of information processing devices, such as a portable wearable device, a smart device, and the like. For example, the various information processing devices may include, but not limited to, wearable devices of various types, such as a smartwatch worn on a wrist, a smart band type, a headphone type, a hairband type, and the like, mobile devices, such as a smartphone, a tablet personal computer (PC), and the like, a professional medical institution system, and the like.

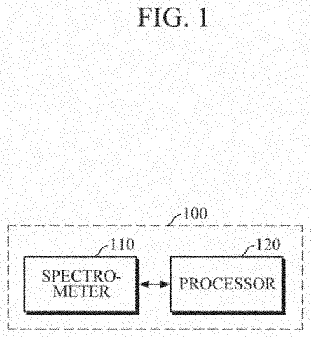

FIG. 1 is a block diagram illustrating an apparatus for estimating bio-information according to an embodiment.

Referring to FIG. 1, the apparatus 100 for estimating bio-information includes a spectrometer 110 and a processor 120.

The spectrometer 110 may measure a spectrum from an object. The spectrometer 110 may measure a spectrum on the basis of Raman spectroscopy or near-infrared (NIR) spectroscopy. The spectrometer 110 may include one or more light sources configured to emit light to the object, and one or more detectors configured to detect light scattered or reflected from the object. The object may include a human skin tissue, or the like, for example, a radial artery region, or an upper wrist part, a finger, or the like, where venous blood or capillary blood passes.

The light source may include a light emitting diode, a laser diode, a phosphor, and the like. The one or more light sources may emit light of different wavelengths. In this case, at least some light sources may have a color filter arranged on an upper part thereof to transmit or block light of a specific wavelength region.

The detector may include one pixel or a pixel array including two or more pixels, in which each pixel may include a photodiode, a phototransistor, a complementary metal-oxide semiconductor (CMOS) image sensor, a charge-coupled device (CCD) image sensor, or the like. Based on detecting light, the detector may convert a detected light signal into an electric signal. A light focusing device, such as a micro-lens, for improving light focusing ability, may be disposed on the top of each pixel.

The processor 120 may be electrically connected to the spectrometer 110, and may control the spectrometer 110 based on a request for estimating bio-information. Based on receiving a signal from the spectrometer 110, the processor 120 may reconstruct a spectrum of the object by using the received signal. The reconstructed spectrum may be used to estimate bio-information from the object. In this case, the bio-information may include, but is not limited to, carotenoids, blood glucose, sugar intake, triglycerides, cholesterol, calories, proteins, body water, extracorporeal water, uric acid, and the like.

Based on the spectrum for estimating bio-information being acquired, the processor 120 may perform various spectrum processing, such as removing noise, caused by a change in external environment, from the acquired spectrum, and the like. In this case, the change in external environment may include various factors, such as temperature, humidity, and the like, which affect accuracy of a spectrum.

The processor 120 may acquire a hemoglobin index and a melanin index using the spectrum acquired through the spectrometer 110, and estimate bio-information by using the acquired hemoglobin index and melanin index.

For example, the processor 120 may acquire the hemoglobin index from the spectrum measured in real-time from the object through the spectrometer 110, and guide a user for spectrum measurement based on the acquired hemoglobin index. For example, the processor 120 may guide the user for a pressure to be applied to the spectrometer 110 based on a correlation between a pressure applied by the object to the spectrometer 110 for spectrometer measurement and the hemoglobin index.

In addition, the processor 120 may acquire the melanin index and the bio-information-related feature from the spectrum finally measured by guiding the spectrum measurement through the hemoglobin index. For example, the processor 120 may estimate the bio-information by applying an estimation model differently defined according to the melanin index to the bio-information related feature.

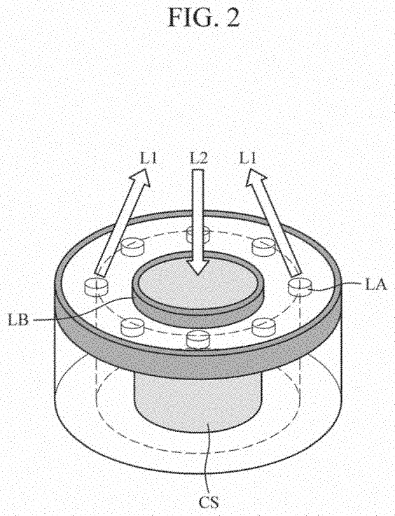

FIG. 2 is a diagram schematically illustrating an embodiment of a structure of a spectrometer. The structure of the spectrometer 110 as shown in FIG. 2 is an embodiment and the structure of the spectrometer is not limited thereto.

Referring to FIG. 2, the spectrometer 110 according to an embodiment may include an array of n number of LED light sources LA arranged on a circular frame. In this case, the shape of the frame is not necessarily limited to a circle and may be modified according to configuration, size, and the like, of the apparatus 100 for estimating bio-information.

Each LED light source may have at least some of peak wavelengths in different wavelength bands. The peak wavelengths of each LED light source may be preset, and may be set based on a spectrum measurement site, a component to be analyzed, and the like. After light is emitted by each of the LED light sources onto the object, the emitted light is absorbed into, or reflected or scattered from, the object depending on tissue properties of the object. In this case, photoreaction properties of the object may vary depending on the types of the object and the wavelengths of light, and the degree of absorption, reflection, transmission, or scattering of light by the object may vary depending on the photoreaction properties of the object. The spectrometer 110 may have a detector CS disposed at the center of the circular frame to detect light L2 scattered or reflected from the object L1 which is irradiated with light L1 by the LED light source LA. Here, the detector CS may be, but not limited to, a CMOS image sensor (CIS)-based sensor, and a spectral filter may be disposed on the CIS to detect light of various wavelengths.

In addition, the spectrometer 110 may include a light blocker LB which blocks the light L1 emitted from the LED light source LA from directly traveling to the detector CS, rather than directing to the object, and directs the light scattered or reflected from the object in the direction of the detector CS.

Based on a spectrum being acquired through the spectrometer 110, the processor 120 may process the spectrum for analyzing bio-information, that is, a component of a body surface or body component, and may estimate bio-information by using the processed spectrum.

The light emitted to the object through the light source of the spectrometer 110 may be absorbed into, or scattered or reflected from, the living tissue of the object, in which case light absorption by hemoglobin in the blood has a great effect on the entire skin light spectrum. Generally, when a spectrum is measured from the object, pressure of a predetermined magnitude or greater may be applied to the object to minimize the light absorption by hemoglobin in the blood. However, the spectrum changes as pressure is applied to the object, and the spectrum dynamically changes depending on the intensity of pressure applied to the object or the time for which the pressure is applied. Therefore, the processor 120 may monitor the change in the pressure being applied to the object as the object presses the spectrometer 110 through the hemoglobin index, which is obtained from the spectrum continuously acquired for a predetermined period of time through the spectrometer 110.

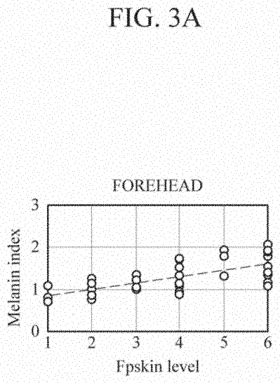

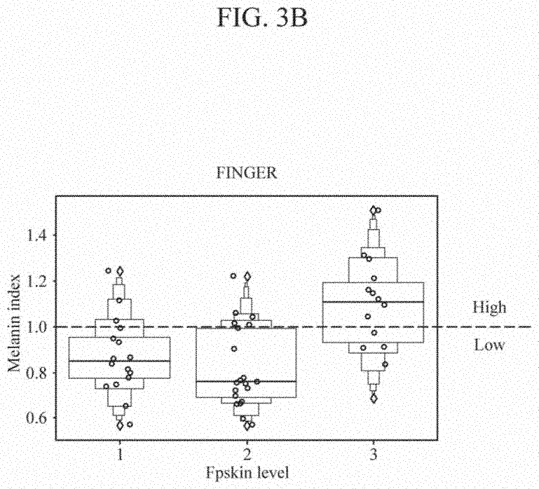

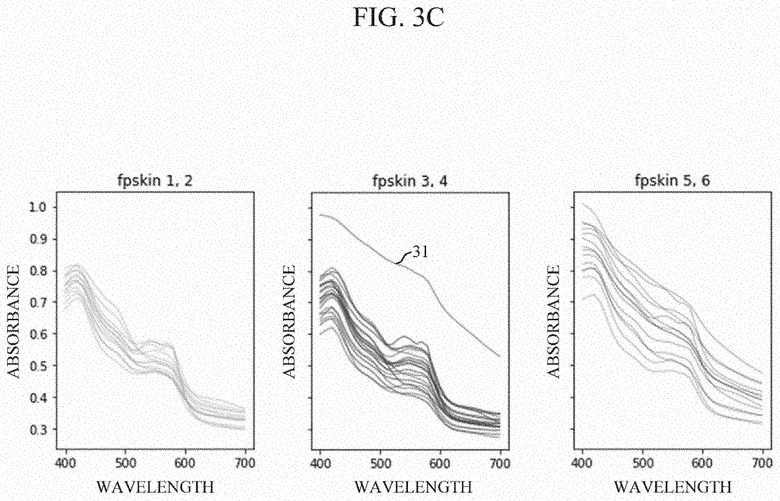

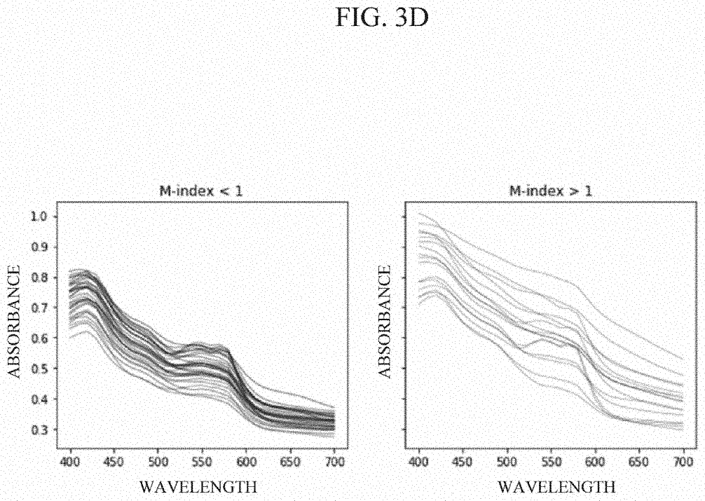

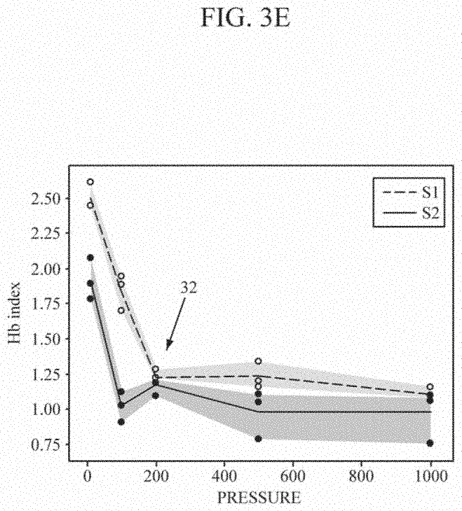

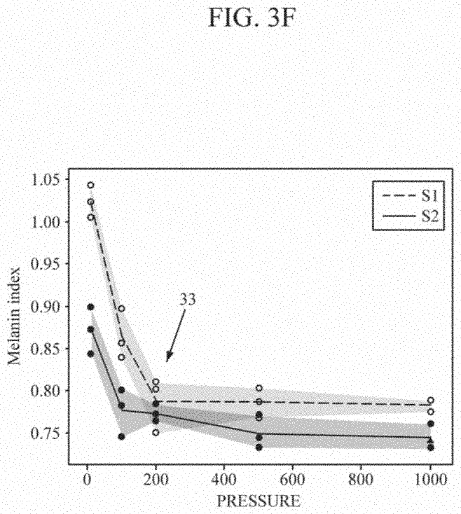

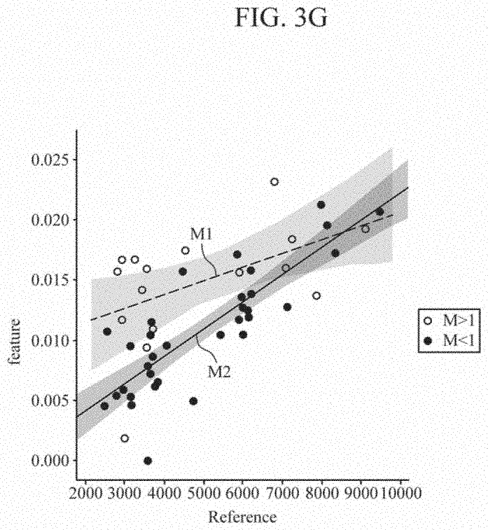

FIGS. 3A to 3G are diagrams for describing a process of estimating bio-information.

An embodiment in which the apparatus 100 for estimating bio-information estimates bio-information will be described with reference to FIGS. 1 to 3G.

FIGS. 3A and 3B illustrate the relationship between a general melanin index and the Fitzpatrick skin type. FIG. 3C illustrates classification results of spectra obtained from multiple people with various skin colors into the Fitzpatrick skin types. FIG. 3D illustrates classification results of spectra obtained from multiple people with various skin colors according to the melanin index.

As shown in FIG. 3A, in the case of measuring a spectrum from a forehead, the melanin index obtained from a spectrum measured from a forehead of each person and the Fitzpatrick skin type (Fpskin level) are generally in a proportional relationship.

However, in the case of measuring a spectrum from a finger, as shown in FIGS. 3B and 3C, not all the Fitzpatrick skin types are in a proportional relationship with melanin indices. In FIG. 3B, value 1 on the X-axis represents the Fitzpatrick skin types 1 and 2, value 2 on the X-axis represents the Fitzpatrick skin types 3 and 4, and value 3 on the X-axis represents the Fitzpatrick skin types 5 and 6.

For example, the melanin index is not proportional to the skin type for the Fitzpatrick skin types 3 and 4 (value 2 on the X-axis) in FIG. 3B. Referring to FIG. 3C, a spectrum 31 is different from other spectra occurs in the graph (middle) of the Fitzpatrick skin types 3 and 4. The abnormal spectrum 31 fits to the Fitzpatrick skin types 5 and 6.

When the Fitzpatrick skin types are applied to classify the spectrum in this manner, a gap may occur between a position at which the Fitzpatrick skin type is to be determined and a spectrum measurement position (e.g., finger, palm, etc.), so that the skin spectrum may be classified differently, which may cause performance degradation depending on the skin type. However, referring to FIG. 3D, spectra obtained from multiple people with various skin colors are more accurately classified when classified into two types on the basis of the melanin index according to the present embodiment Thus, when the bio-information is estimated using the spectrum classified in this manner, the accuracy may be improved.

FIG. 3E illustrates a relationship between the hemoglobin indices acquired from skin spectra of two samples S1 and S2 and the pressure applied to the spectrometer 110 to acquire each skin spectrum. FIG. 3F illustrates a relationship between the melanin indices acquired from skin spectra of two samples S1 and S2 and the pressure applied to the spectrometer 110 to acquire each skin spectrum. As shown in FIG. 3E, the light absorption by hemoglobin gradually decreases as the pressure steadily increases, whereby the hemoglobin index rapidly decreases to a specific point 31, and then the decreasing rate is gradually reduced. Likewise, as shown in FIG. 3F, the melanin index rapidly decreases to a specific point 32 as the pressure gradually increases, and then the decreasing rate is reduced.

Thus, the processor 120 may guide the measurement pressure between the object and the spectrometer 110 while measuring the spectrum using the relationship between the hemoglobin index and the pressure. That is, the user may be guided to maintain a predetermined pressure so that the light absorption by hemoglobin in the blood is minimized during the spectrum measurement.

For example, based on receiving spectrum data in real-time from the spectrometer 110, the processor 120 may acquire the hemoglobin index from the received spectrum. In this case, various known techniques may be used to acquire the hemoglobin index from the spectrum.

Also, the processor 120 may compare the acquired hemoglobin index with a predetermined threshold and guide the user to maintain a specific level of pressure during the spectrum measurement. Here, the predetermined threshold may be preset for each user through preprocessing. For example, referring to FIG. 3E, in the case of the skin spectrum of sample S1, a hemoglobin index value of about 1.25 at the point 31 at which the decreasing rate of the hemoglobin index rapidly decreasing with the increase of the pressure is reduced may be set to the predetermined threshold. That is, in FIG. 3E, the processor may guide the user to apply a pressure of about 200 or greater to the spectrometer 110 so that the hemoglobin index acquired in real-time from the spectrum is maintained to be less than or equal to the threshold of 1.25.

FIG. 3G illustrates an estimation model defined by the processor 120 according to the melanin index. In the graph of FIG. 3G, the X-axis represents a reference melanin index acquired from spectra of a plurality of users through an external device and the Y-axis represents beta-carotene feature values acquired from the plurality of users.

Based on the final spectrum data being measured by the spectrometer 110, the processor 120 may acquire the melanin index from the spectrum data. In this case, various known techniques may be used to acquire the melanin index from the spectrum.

Further, the processor 120 may acquire a bio-information-related feature from the final spectrum. Here, the bio-information-related feature may include, for example, beta-carotene feature, which may be acquired using Equation 1 below. However, Equation 1 is merely an example.

$$f = ( {A}_{λ2} - \frac{{A}_{λ1} + {A}_{λ3}}{2} ) \qquad [ Equation 1 ]$$

In Equation 1, A_{λ1} denotes absorbance of wavelength λ1, A_{λ2} denotes absorbance of wavelength λ2, and A_{λ3} denotes absorbance of wavelength λ3, wherein λ2 is a mean wavelength of wavelengths λ1 and λ3.

Based on acquiring the bio-information-related features from the spectrum, the processor 120 may estimate bio-information by applying the estimation model, predefined according to the acquired melanin index, to the bio-information-related feature. In this case, two or more estimation models may be defined based on the melanin index. Also, the estimation model may be defined as a linear function equation, but is not limited thereto. For example, referring to FIG. 3G, the processor 120 may apply a first estimation model M1 to the acquired beta-carotene feature when the melanin index acquired from the spectrum of the user is greater than or equal to 1, and may apply a second estimation model M2 to the beta-carotene feature when the melanin index is less than 1.

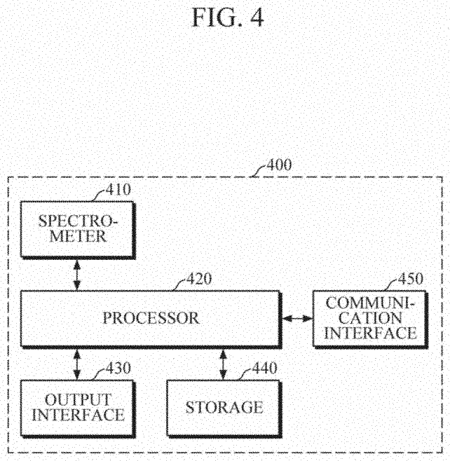

FIG. 4 is a block diagram illustrating an apparatus for estimating bio-information according to another embodiment.

Referring to FIG. 4, the apparatus 400 for estimating bio-information may include a spectrometer 410, a processor 420, an output interface 430, a storage 440, and a communication interface 450. The spectrometer 410 and the processor 420 have already been described in detail above, such that redundant description will be omitted.

The output interface 430 may output various types of information processed by the processor 420. The output interface 430 may include a visual output module, such as a display, or the like, a voice output module, such as a speaker, or the like, or a haptic module providing, for example, vibration or tactile sensation. The processor 420 may control the output interface 430 to output guide information to guide the user regarding spectrum measurement.

For example, based on the processor 420 acquiring the hemoglobin index from the spectrum as described above and guiding the measurement pressure based on the acquired hemoglobin index, the output interface 430 may output guide information generated by the processor 420. For example, the output interface 430 may visually display a graph showing an actual measurement pressure converted based on the acquired hemoglobin index on a display. In this case, a reference pressure to be applied by the user to the spectrometer 410 may be displayed together as a graph. In addition, the output interface 430 may output a voice signal, tactile sensation, vibration, or the like to the user based on the actual measurement pressure not being within the reference pressure range.

Further, the output interface 430 may output a final spectrum and an acquired bio-information estimate value by using the final spectrum. In this case, the output interface 430 may divide the display into two or more sections, and display the bio-information estimate value on a first section and display detailed information used to estimate bio-information, for example, the final spectrum, the melanin index, the hemoglobin index, the measurement pressure, the health condition, and the like, on a second section.

The storage 440 may store user information, light source driving conditions, an estimation model, the hemoglobin index, and reference information, such as a threshold that is a criterion for comparison. Further, the spectrum measured by the spectrometer 410 and/or the processing result of the processor 420, for example, information such as the hemoglobin index, the melanin index, and the bio-information estimate value may be stored.

The storage 440 may include at least one storage medium of a flash memory type memory, a hard disk type memory, a multimedia card micro type memory, a card type memory (e.g., a secure digital (SD) memory, an extreme digital (XD) memory, etc.), a random access memory (RAM), a static random access memory (SRAM), a read only memory (ROM), an electrically erasable programmable read only memory (EEPROM), a programmable read only memory (PROM), a magnetic memory, a magnetic disk, and an optical disk, but is not limited thereto.

The communication interface 450 may communicate with an external device through wired or wireless communication, and receive various types of information from the external device. The external device may include an information processing device, such as a smartphone, a tablet PC, a notebook computer, a desktop computer, or the like, but is not limited thereto.

For example, the communication interface 450 may receive a request for measuring a spectrum from the external device and transmit the request to the processor 420. The communication interface 450 may receive reference information, such as conditions for driving the light source, the estimation model, and the like. Also, the communication interface 450 may transmit the spectrum measured by the spectrometer 410, the hemoglobin index, melanin index, bio-information estimate value, and the like, which are acquired by the processor 420, to the external device.

Further, the communication interface 450 may communicate with the external device by using Bluetooth communication, Bluetooth low energy (BLE) communication, near field communication (NFC), wireless local access network (WLAN) communication, ZigBee communication, infrared data association (IrDA) communication, wireless fidelity (Wi-Fi) Direct (WFD) communication, ultra-wideband (UWB) communication, Ant+ communication, Wi-Fi communication, radio frequency identification (RFID) communication, 3G communication, 4G communication, and/or 5G communication. However, these are merely examples, and the embodiment is not limited thereto.

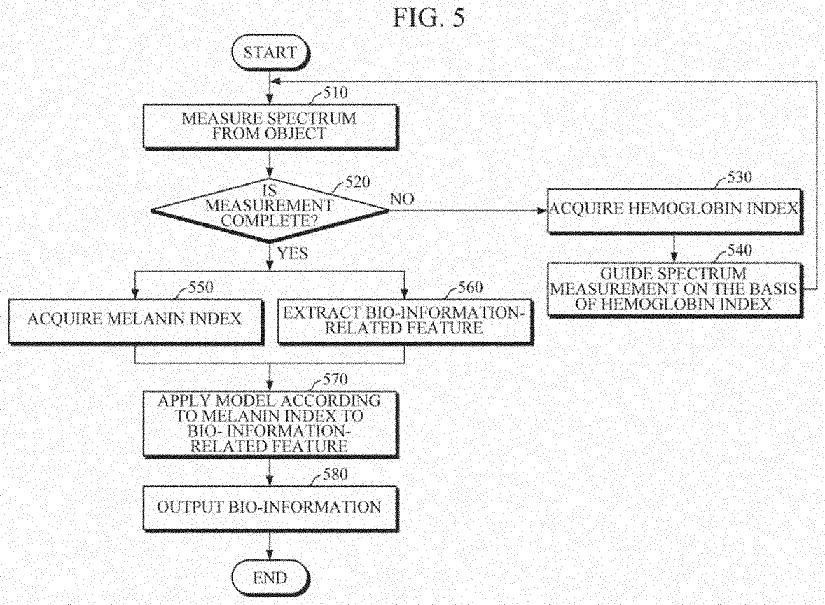

FIG. 5 is a flowchart illustrating a method of estimating bio-information according to an embodiment.

Referring to FIG. 5, the apparatus for estimating bio-information 100/400 may measure a spectrum in response to a request for measuring a spectrum of an object (operation 510). The request for measuring a spectrum may be generated according to a user's input, a preset period, a request from an external device, or the like.

Also, the apparatus 100/400 for estimating bio-information may determine whether the spectrum measurement is complete (operation 520), and based on the spectrum measurement being in progress, may acquire a hemoglobin index from the measured spectrum (operation 530).

In addition, the apparatus 100/400 may guide the user for spectrum measurement by using the acquired hemoglobin index (operation 540). For example, as described above, a measurement pressure may be guided to the user by using the relationship between a pressure applied by the spectrometer to the object and the hemoglobin index, such that the hemoglobin index is maintained to be lower than or equal to a predetermined threshold. Operations 520, 530, and 540 may continue while the spectrum measurement is in progress in operation 510.

If it is determined in operation 520 that the spectrum measurement is complete (operation 520—YES), a melanin index may be acquired from the measured final spectrum (operation 550), and a bio-information-related feature may also be acquired (operation 560). In this case, the bio-information related feature may be predefined according to the bio-information to be estimated, and may be, for example, a feature related to beta-carotene.

Then, an estimation model predefined according to the melanin index acquired in operation 550 is applied to the bio-information related feature acquired in operation 550 to estimate bio-information (operation 570). Two or more estimation models may be defined according to the melanin index, and may include a personalized model for each user or a generic estimation model applicable to a plurality of users.

Then, the estimated bio-information may be output (operation 580). The bio-information estimate value may be visually output through a display and warning information, or the like, may be non-visually provided to the user in voice or through vibration, tactile sensation, or the like.

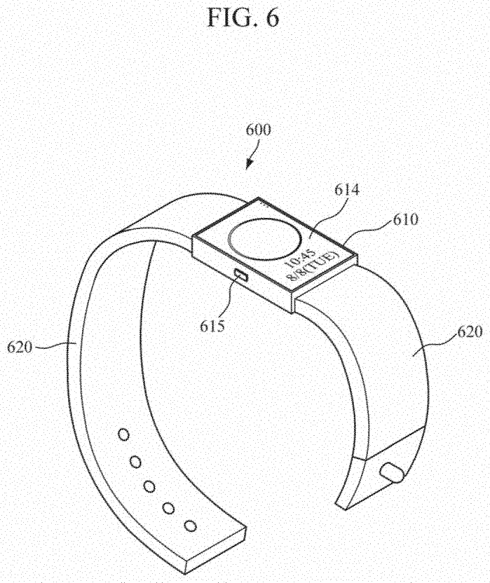

FIG. 6 is a diagram illustrating a wearable device according to an embodiment.

The wearable device 600 illustrated in FIG. 6 may be a smart watch, but is not limited thereto. Various embodiments of the apparatus 100/400 for estimating bio-information described above may be mounted in the wearable device 600.

Referring to FIG. 6, the wearable device 600 may include a main body 610 and a strap 620.

The main body 610 may be provided in various shapes, may include modules for performing general functions of the wearable device 600, and be equipped with a function for estimating bio-information. A battery may be embedded in the main body 610 or the strap 620 to supply power to various modules.

The strap 620 may be connected to the main body 610. The strap 620 may be flexible so as to be bent around a user's wrist. The strap 620 may include a first strap and a second strap that is separated from the first strap. Respective ends of the first and second straps are connected to both sides of the main body MB, and the first strap and the second strap may be fastened to each other using fastening means formed on the other sides thereof. In this case, the fastening means may be formed as Velcro fastening, pin fastening, or the like, but is not limited thereto. In addition, the strap ST may be formed as an integrated piece, such as a band.

Further, the wearable device 600 may include a spectrometer and a processor. The spectrometer may be disposed on a rear surface of the main body 610 that comes into contact with an upper portion of the user's wrist when the main body 610 is wom on the user's wrist. In addition, the processor may be mounted in the main body 610 and electrically connected to the spectrometer.

The spectrometer may include a light source formed as an LED array including a plurality of LEDs and a detector as shown in FIG. 2. The spectrometer may drive the light source to emit light to the user's skin in response to a control signal of the processor and acquire a spectrum by detecting light returning through the user's skin. In this case, the light source may be configured to emit light of near-infrared wavelengths or mid-infrared wavelengths. The spectrometer may include a linear variable filter (LVF). The LVF may have a spectral characteristic in which the filter linearly changes over the entire length. Accordingly, the linear variable filter may disperse incident light by wavelength. The linear variable filter has a compact size but an excellent spectral capability.

The processor may control the spectrometer based on a user's request or a predefined criterion being satisfied. The processor may acquire a hemoglobin index from a spectrum while the spectrum is being measured by the spectrometer, and may guide the user for a measurement pressure by using the acquired hemoglobin index. Further, based on the spectrum measurement being complete, the processor may acquire a melanin index and bio-information-related feature from the measured spectrum, and estimate bio-information by applying an estimation model defined according to the melanin index to the acquired bio-information-related feature.

The wearable device 600 may further include a manipulator 615 and a display 614. The manipulator 615 may be formed on a stem on a side of the main body 610 as illustrated. The manipulator 615 may receive a command of a user and transmit the received command to the processor, and may include a power button for turning on/off the wearable device 600.

The display 614 may display information, such as a bio-information estimate value, a warning, or the like, in various visual ways under the control of the processor and provide the information to the user.

In addition, the wearable device 600 may include a communication interface. The communication interface may communicate with an external device, such as a smartphone, a tablet PC, a desktop computer, a notebook computer, or the like, to transmit and receive various types of data.

The embodiments can be implemented as computer readable code stored in a non-transitory computer-readable medium that is executed by a processor. Code and code segments constituting the computer program can be inferred by a skilled computer programmer in the art. The computer-readable medium includes all types of recording media in which computer-readable data is stored. Examples of the computer-readable medium include a ROM, a RAM, a CD-ROM, a magnetic tape, a floppy disk, and an optical data storage. Further, the computer-readable medium may be implemented in the form of a carrier wave such as Internet transmission. In addition, the computer-readable medium may be distributed to computer systems over a network, in which computer-readable code may be stored and executed in a distributed manner.

A number of examples have been described above. Nevertheless, it will be understood that various modifications may be made. For example, suitable results may be achieved if the described techniques are performed in a different order and/or if components in a described system, architecture, device, or circuit are combined in a different manner and/or replaced or supplemented by other components or their equivalents. Accordingly, other implementations are within the scope of the following claims.

What is claimed is:

1. An apparatus for estimating bio-information of a user, the apparatus comprising:
a spectrometer configured to measure a spectrum from an object of the user; and
a processor configured to:
control an output interface to output guide information to guide the user regarding spectrum measurement based on a hemoglobin index of the user while the spectrum is being measured by the spectrometer; and
estimate the bio-information of the user based on a melanin index of the user and the spectrum.

2. The apparatus of claim 1, wherein the processor is further configured to acquire the hemoglobin index of the user from the spectrum while the spectrum is being measured.

3. The apparatus of claim 2, wherein the processor is further configured to control the output interface to output the guide information to guide the user regarding a measurement pressure between the object and the spectrometer based on the acquired hemoglobin index.

4. The apparatus of claim 3, wherein the processor is further configured to control the output interface to output the guide information to guide the user regarding the measurement pressure based on a predefined relationship between the measurement pressure and the hemoglobin index such that the hemoglobin index is maintained to be less than or equal to a predetermined threshold.

5. The apparatus of claim 1, wherein the processor is further configured to, based on the spectrum being measured, acquire the melanin index from the spectrum.

6. The apparatus of claim 5, wherein the processor is further configured to, based on the spectrum being measured, acquire a bio-information-related feature from the spectrum.

7. The apparatus of claim 6, wherein the processor is further configured to estimate the bio-information by applying a predetermined model to the bio-information-related feature based on the melanin index.

8. The apparatus of claim 7, wherein the processor is further configured to apply a first model to the bio-information-related feature based on the melanin index being greater than or equal to a predetermined threshold, and apply a second model to the bio-information-related feature based on the melanin index being less than the predetermined threshold.

9. The apparatus of claim 1, wherein the spectrometer comprises one or more light sources configured to emit light to the object, and one or more detectors configured to detect light scattered or reflected from the object.

10. The apparatus of claim 1, further comprising the output interface configured to output a processing result of the processor.

11. The apparatus of claim 1, further comprising a communication interface configured to transmit a processing result of the processor to an external device.

12. The apparatus of claim 1, wherein the bio-information includes one or more of carotenoids, blood glucose, sugar intake, triglycerides, cholesterol, calories, proteins, body water, extracorporeal water, and uric acid.

13. A method of estimating bio-information of a user, the method comprising:
measuring a spectrum from an object of the user;
guiding a user regarding spectrum measurement based on a hemoglobin index of the user while the spectrum is being measured; and
estimating the bio-information of the user based on a melanin index of the user and the spectrum.

14. The method of claim 13, further comprising acquiring the hemoglobin index from the spectrum while the spectrum is being measured.

15. The method of claim 14, wherein the guiding the user regarding the spectrum measurement comprises guiding the user regarding a measurement pressure between the object and the spectrometer based on the acquired hemoglobin index.

16. The method of claim 15, wherein the guiding the user regarding the spectrum measurement comprises guiding the user regarding the measurement pressure based on a predefined relationship between the measurement pressure and the hemoglobin index such that the hemoglobin index is maintained to be less than or equal to a predetermined threshold.

17. The method of claim 13, further comprising, based on the spectrum being measured, acquiring the melanin index from the spectrum.

18. The method of claim 17, further comprising, based on the spectrum being measured, acquiring a bio-information-related feature from the spectrum.

19. The method of claim 18, wherein the estimating of the bio-information of the user comprises estimating the bio-information of the user by applying a predetermined model to the bio-information-related feature according to the melanin index.

20. The method of claim 19, wherein the estimating of the bio-information of the user comprises applying a first model to the bio-information-related feature based on the melanin index being greater than or equal to a predetermined threshold, and, applying a second model to the bio-information-related feature based on the melanin index being less than the predetermined threshold.
